(19) United States
(12) Patent Application Publication
(10) Pub. No.: US 2020/0372990 A1
(43) Pub. Date: Nov. 26, 2020

(54) Systems and Methods for Visualizing and Modifying Treatment of a Patient Utilizing a Digital Therapeutic

(71) Applicant: Pear Therapeutics, Inc., Boston, MA (US)

(72) Inventor: Andrew P. Ho, San Mateo, CA (US)

(73) Assignee: Pear Therapeutics, Inc., Boston, MA (US)

(21) Appl. No.: 16/881,158

(22) Filed: May 22, 2020

Related U.S. Application Data
(60) Provisional application No. 62/851,304, filed on May 22, 2019.

(51) Int. Cl. G16H 20/10; G06F 16/26; G16H 10/60; G16H 15/00; G16H 50/20; A61B 5/00; A61B 5/16; G06F 40/166; G06F 3/0482

(52) U.S. Cl. CPC G16H 20/10; G06F 16/26; G16H 10/60; G16H 15/00; G16H 50/20; G06F 3/0482; A61B 5/742; A61B 5/165; A61B 5/4806; A61B 5/7264; G06F 40/166; A61B 5/4848

(57) ABSTRACT

A system includes data processing hardware and memory hardware in communication with the data processing hardware, the memory hardware storing instructions that when executed on the data processing hardware cause the data processing hardware to perform operations comprising: (i) obtaining input data from data processing hardware of a patient electronic device associated with a patient; (ii) generating and displaying on a display a graphical interface based on the input data; (iii) receiving a user input selection indicating selection of one of the first graphical element or the second graphical element; and (iv) modifying the selected first or second graphical element based on the user input selection.

### CROSS REFERENCE TO RELATED APPLICATIONS

This U.S. patent application claims priority under 35 U.S.C. §119(e) to U.S. Provisional Application 62/851,304, filed on May 22, 2019. The disclosure of this prior application is considered part of the disclosure of this application and is hereby incorporated by reference in its entirety.

### FIELD

This disclosure relates, generally, to the treatment of serious medical conditions and, more particularly, to systems and methods for visualizing and modifying treatment of a patient utilizing a digital therapeutic.

### BACKGROUND

The information provided in this section is for the purpose of generally presenting the context of the disclosure. Work of the presently named inventor, to the extent it is described in this section, as well as aspects of the description that may not otherwise qualify as prior art at the time of filing, are neither expressly nor impliedly admitted as prior art against the present disclosure.

Drug therapy has played a significant role in the treatment of various medical diseases and disorders. Traditional drug therapy involves the administration of pharmaceuticals and the like. Examples of conventional pharmaceuticals may include small-molecule drugs, which are usually derived from chemical synthesis, and biopharmaceuticals, which may include recombinant proteins, vaccines, blood products used therapeutically gene therapy, monoclonal antibodies, cell therapy, and the like.

While drug therapy has proven to be an effective mechanism for treating certain diseases and disorders, it is not without drawbacks. For example, pharmaceuticals are known to come with certain, frequently undesirable, side-effects. In addition, pharmaceuticals are often costly—sometimes prohibitively so.

Accordingly, digital solutions for treating various medical diseases and disorders have emerged as a compliment, or alternative, to conventional drug therapy techniques. Such digital solutions (e.g., digital therapeutics, mobile health applications, etc.) may solicit information from users (e.g., patients in the case of a prescription digital therapeutic or “PDT”) thereof. Such information may include, by way of example and not limitation, information concerning the user's mental state (e.g., feelings the user is or has experienced) and/or physical state (e.g., physical symptoms associated with mental or physical health conditions).

One example of a method of documentation frequently used by healthcare providers is the Subjective, Objective, Assessment, and Plan (SOAP) note. In addition to providing healthcare providers with a cognitive framework for clinical reasoning, SOAP notes are also helpful communication documents between separate healthcare providers and for a single healthcare provider monitoring a patient over an extended period of time. For example, SOAP notes may help a healthcare provider track the progress of a patient suffering from a medical condition causing various mental and/or physical symptoms.

Under the “subjective” heading of a SOAP note, personal views or feelings of the patient or someone close to them is documented. Under the “objective” heading, data such as, for example, vital signs, physical exam findings, laboratory data, and medication adherence, are documented. Under the “assessment” heading, a diagnosis, analysis and/or assessment of the patient's status is documented based on, for example, information found under the “subjective” heading and the “objective” heading. Under the “plan” heading, a treatment plan is documented.

Large amounts of data may be collected from a variety of sources pertaining to a specific patient. Because healthcare providers are limited in the amount of time they can devote to each individual patient, systems and methods for efficiently visualizing and modifying treatment of a patient utilizing a digital therapeutic are desired.

### SUMMARY

One aspect of the disclosure provides a system comprising data processing hardware and memory hardware in communication with the data processing hardware, the memory hardware storing instructions that when executed on the data processing hardware cause the data processing hardware to perform operations including obtaining input data from data processing hardware of a patient electronic device associated with a patient, the input data corresponding to a subjective, objective, assessment, and plan (SOAP) note. The operations include obtaining medication dosage data corresponding to a current medication dosage prescribed to the patient. The operations include generating and displaying on a display a graphical interface based on the input data and the medication dosage data, the graphical interface comprising: (a) a first graphical element displaying a first subset of the input data, the first subset of the input data corresponding to a first duration of time; and (b) a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed. The operations include analyzing the input data and the medication dosage data to generate modified medication dosage data corresponding to a modified medication dosage prescribed to the patient. The operations include modifying the current medication dosage prescribed to the patient based on the modified medication dosage data.

Implementations of the disclosure may include one or more of the following optional features. In some implementations, the input data includes active data input to the patient electronic device by the patient and passive data collected by the patient electronic device. The input data may include at least one of medication utilization data, application utilization data, efficacy data, mood data, productivity data, skill areas data, or sleep data.

The operations may further comprise analyzing the input data and the medication dosage data to generate change medication data corresponding to a change of the medication prescribed to the patient, and modifying the current medication dosage prescribed to the patient based on the change medication data.

At least a portion of the input data may be generated by a healthcare provider associated with the patient. The modified medication dosage data may be generated by implementing artificial intelligence. The graphical interface may further comprise an add entry element configured to facilitate entry of free text data by a healthcare provider associated with the patient. The patient electronic device may be one of a desktop computer, a laptop computer, a tablet computer, a smartphone, or a wearable device.

Another aspect of the disclosure provides a system comprising data processing hardware and memory hardware in communication with the data processing hardware, the memory hardware storing instructions that when executed on the data processing hardware cause the data processing hardware to perform operations including obtaining input data from data processing hardware of a patient electronic device associated with a patient. The operations include generating and displaying on a display a graphical interface based on the input data, the graphical interface comprising: (a) a first graphical element displaying a first subset of the input data, the first subset of the input data corresponding to a first duration of time; and (b) a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed. The operations include receiving a user input selection indicating selection of one of the first graphical element or the second graphical element. The operations include modifying the selected first or second graphical element based on the user input selection. This aspect may include one or more of the following optional features.

The input data may include active data input to the patient electronic device by the patient and passive data collected by the patient electronic device. The input data includes at least one of medication utilization data, application utilization data, efficacy data, mood data, productivity data, skill areas data, or sleep data. The input data may correspond to a subjective, objective, assessment, and plan (SOAP) note. At least a portion of the input data is generated by a healthcare provider associated with the patient. At least a portion of the input data is generated by a mental health provider associated with the patient.

The graphical interface may further comprise an add entry element configured to facilitate entry of free text data by a healthcare provider associated with the patient.

Another aspect of the disclosure provides a method including obtaining, via one or more processors, input data from data processing hardware of a patient electronic device associated with a patient, the input data corresponding to a subjective, objective, assessment, and plan (SOAP) note. The method includes obtaining, via the one or more processors, medication dosage data corresponding to a current medication dosage prescribed to the patient. The method includes generating and displaying, via the one or more processors, on a display a graphical interface based on the input data and the medication dosage data, the graphical interface comprising: (a) a first graphical element displaying a first subset of the input data, the first subset of the input data corresponding to a first duration of time; and (b) a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed. The method includes analyzing, via the one or more processors, the input data and the medication dosage data to generate modified medication dosage data corresponding to a modified medication dosage prescribed to the patient. The method includes modifying, via the one or more processors, the current medication dosage prescribed to the patient based on the modified medication dosage data. This aspect may include one or more of the following optional features.

The input data may include active data input to the patient electronic device by the patient and passive data collected by the patient electronic device. The input data may include at least one of medication utilization data, application utilization data, efficacy data, mood data, productivity data, skill areas data, or sleep data. At least a portion of the input data may be generated by a healthcare provider associated with the patient. The modified medication dosage data may be generated by implementing artificial intelligence.

Another aspect of the disclosure provides a method including obtaining, via one or more processors, input data from data processing hardware of a patient electronic device associated with a patient. The method includes generating and displaying, via the one or more processors, on a display a graphical interface based on the input data, the graphical interface comprising: (a) a first graphical element displaying a first subset of the input data, the first subset of the input data corresponding to a first duration of time; and (b) a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed. The method includes receiving, via the one or more processors, a user input selection indicating selection of one of the first graphical element or the second graphical element. The method includes modifying, via the one or more processors, the selected first or second graphical element based on the user input selection.

The details of one or more implementations of the disclosure are set forth in the accompanying drawings and the description below. Other aspects, features, and advantages will be apparent from the description and drawings, and from the claims.

### BRIEF DESCRIPTION OF THE FIGURES

Reference will now be made to the accompanying Figures, which are not necessarily drawn to scale, and wherein:

FIG. 1 is a schematic view of a system for displaying and managing patient data including a healthcare provider application in accordance with an exemplary embodiment of the present disclosure;

FIG. 2A illustrates an exemplary subjective graphical user interface (GUI) displayed on a healthcare provider (HCP) system in accordance with an exemplary embodiment of the present disclosure;

FIG. 2B illustrates another exemplary subjective GUI displayed on HCP system in accordance with an exemplary embodiment of the present disclosure;

FIG. 3A illustrates an exemplary objective GUI displayed on HCP system in accordance with an exemplary embodiment of the present disclosure;

FIG. 3B illustrates another exemplary objective GUI displayed on HCP system in accordance with an exemplary embodiment of the present disclosure;

FIG. 4 illustrates an exemplary assessment GUI displayed on HCP system in accordance with an exemplary embodiment of the present disclosure;

FIG. 5 illustrates an exemplary plan GUI displayed on HCP system in accordance with an exemplary embodiment of the present disclosure;

FIG. 6 illustrates a flowchart showing a method for implementing the system of FIG. 1;

FIG. 7 illustrates a flowchart showing another method for implementing the system of FIG. 1; and

FIG. 8 is a schematic view of an example computing device that may be used to implement the systems and methods described herein in accordance with an exemplary embodiment of the present disclosure.

Like reference symbols in the various drawings indicate like elements.

### DETAILED DESCRIPTION

Some implementations of the disclosed technology will be described more fully with reference to the accompanying drawings. This disclosed technology may, however, be embodied in many different forms and should not be construed as limited to the implementations set forth herein.

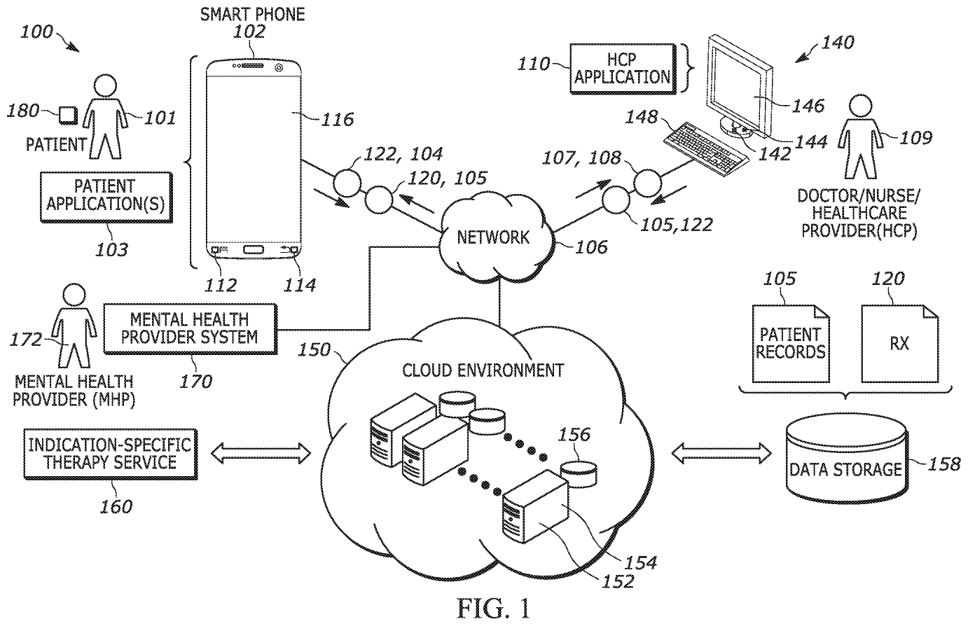

Referring to FIG. 1, in some implementations, a therapy prescription system 100 provides a patient 101 access to a prescription digital therapeutic 120 prescribed to the patient 101 and monitors events associated with the patient's 101 interaction with the prescription digital therapeutic 120. Although the digital therapeutic 120 is described herein as being a “prescription” digital therapeutic, it is understood that, according to some implementations, the digital therapeutic 120 will not require a prescription from a clinician. Rather, in such implementations, the digital therapeutic 120 may be available to a patient without a prescription, and the digital therapeutic 120 nonetheless otherwise functions in accordance with the description of the prescription digital therapeutic 120 described herein. According to implementations in which the digital therapeutic 120 is not prescribed, the person using or being administered the digital therapeutic may be referred to as a “user.” A “user” may include a patient 101 or any other person using or being administered the digital therapeutic 120, irrespective of whether the digital therapeutic 120 was prescribed to that person.

As used herein, a digital therapy may also be referred to as a digital-therapeutic configured to deliver evidence-based psychosocial intervention techniques for treating a patient with a particular disease or disorder, as well as symptoms and/or behaviors associated with the particular disease or disorder. As one example, the patient 101 may be diagnosed with a chronic disease and the prescription digital therapeutic 120 may be specifically tailored for addressing one or more symptoms associated with the chronic disease that the patient 101 may experience. An authorized healthcare provider (HCP) 109 (e.g., a doctor, nurse, etc.) supervising the patient 101 may prescribe the patient 101 the prescription digital therapeutic 120 designed to help the patient 101 identify feelings the patient 101 is experiencing and modify dysfunction emotions, behaviors, and thoughts in order to treat symptoms in the patient 101. The HCP 109 may include a physician, nurse, clinician, or other qualified health professionals.

In some examples, the system 100 includes a network 106, a patient device 102, an HCP system 140, and a medical indication-specific therapy service 160. For example, the therapy service 160 may be related to a specific indication such as opioid abuse, multiple sclerosis, depression, etc. In some implementations, the system 100 may include a mental health provider (MHP) system 170. The network 106 provides access to cloud computing resources 150 (e.g., distributed system) that execute the therapy service 160 to provide for the performance of services on remote devices. Accordingly, the network 106 allows for interaction between patients 101 and HCPs 109 with the therapy service 160. For instance, the therapy service 160 may provide the patient 101 access to the prescription digital therapeutic 120 and receive event data 122 inputted by the patient 101 associated with the patient's 101 interaction with the prescription digital therapeutic 120. In turn, the therapy service 160 may store the event data 122 on a storage resource 156.

The network 106 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, a satellite communications network, and other communication networks. The network 106 may include one or more of a Wide Area Network (WAN), a Local Area Network (LAN), and a Personal Area Network (PAN). In some examples, the network 106 includes a combination of data networks, telecommunication networks, and a combination of data and telecommunication networks. The patient device 102, the HCP system 140, the therapy service 160, and, in some implementations, the MHP system 170 communicate with each other by sending and receiving signals (wired or wireless) via the network 106, which, in some examples, may utilize Bluetooth, Wi-Fi, etc. In some examples, the network 106 provides access to cloud computing resources, which may be elastic/on-demand computing and/or storage resources 156 available over the network 106. The term “cloud” services generally refers to a service performed not locally on a user's device, but rather delivered from one or more remote devices accessible via one or more networks 106.

The patient device 102 may include, but is not limited to, a portable electronic device (e.g., smartphone, cellular phone, personal digital assistant, personal computer, wireless tablet device, or a wearable device such as a wristband, a heart rate monitor, a sleep monitor, etc.), a desktop computer, or any other electronic device capable of sending and receiving information via the network 106. The patient device 102 includes data processing hardware 112 (a computing device that executes instructions), memory hardware 114, and a display 116 in communication with the data processing hardware 112. In some examples, the patient device 102 includes a keyboard, mouse, microphones, and/or a camera for allowing the patient 101 to input data. In addition to or in lieu of the display 116, the patient device 102 may include one or more speakers to output audio data to the patient 101. For instance, audible alerts may be output by the speaker to notify the patient 101 about some time sensitive event associated with the prescription digital therapeutic 120. In some implementations, the patient device 102 may be connected (e.g., wired or wirelessly) to one or more sensors 180 configured to generate sensor data associated with the patient 101. For example, the one or more sensors 180 may include a heart rate monitor (pulse/EKG), an accelerometer, a hydration sensor, a glucose sensor, a blood pressure sensor, a temperature sensor, a weight sensor, etc.

In some implementations, the patient device 102 executes a patient application 103 (or accesses a web-based patient application) for establishing a connection with the therapy service 160 to access the prescription digital therapeutic 120. For instance, the patient 101 may have access to the patient application 103 for a duration (e.g., 3 months) of the prescription digital therapeutic 120 prescribed to the patient 101. Here, the patient device 102 may launch the patient application 103 by initially providing an access code 104 when the prescription digital therapeutic 120 is prescribed by the HCP 109 that allows the patient 101 to access content associated with the prescription digital therapeutic 120 from the therapy service 160 that is specifically tailored for treating/addressing one or more symptoms associated with the specific indication that the patient 101 may be experiencing. The patient application 103, when executing on the data processing hardware 112 of the patient device 102, is configured to display a variety of graphical user interfaces (GUIs) on the display 116 of the patient device 102 that, among other things, allow the patient 101 to input event data 122 associated with particular feelings the patient is experiencing, solicit information from the patient 101, and present journal entries for the patient 101 to view.

The storage resources 156 may provide data storage 158 for storing the event data 122 received from the patient 101 in a corresponding patient record 105 as well as the prescription digital therapeutic 120 prescribed to the patient 101. The patient record 105 may be encrypted while stored on the data storage 158 so that any information identifying patient 101 is anonymized, but may later be decrypted when the patient 101 or supervising HCP 109 requests the patient record 105 (assuming the requester is authorized/authenticated to access the patient record 105). All data transmitted over the network 106 between the patient device 102 and the cloud computing system 150 may be encrypted and sent over secure communication channels. For instance, the patient application 103 may encrypt the event data 122 before transmitting to the therapy service 160 via the HTTPS protocol and decrypt a patient record 105 received from the therapy service 160. When network connectivity is not available, the patient application 103 may store the event data 122 in an encrypted queue within the memory hardware 114 until network connectivity is available.

The HCP system 140 may be located at a clinic, doctor's office, or facility administered by the HCP 109 and includes data processing hardware 142, memory hardware 144, and a display 146. The memory hardware 144 and the display 146 are in communication with the data processing hardware 142. For instance, the data processing hardware 142 may reside on a desktop computer or portable electronic device for allowing the HCP 109 to input and retrieve data to and from the therapy service 160. In some examples, the HCP 109 may initially onboard some or all of patient data 107 at the time of prescribing the prescription digital therapeutic 120 to the patient 101. The HCP system 140 includes a keyboard 148, mouse, microphones, speakers and/or a camera.

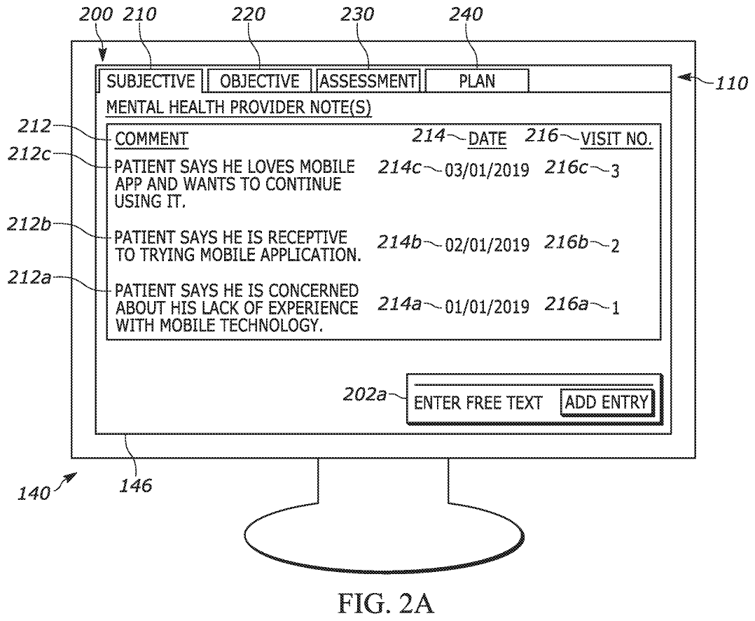

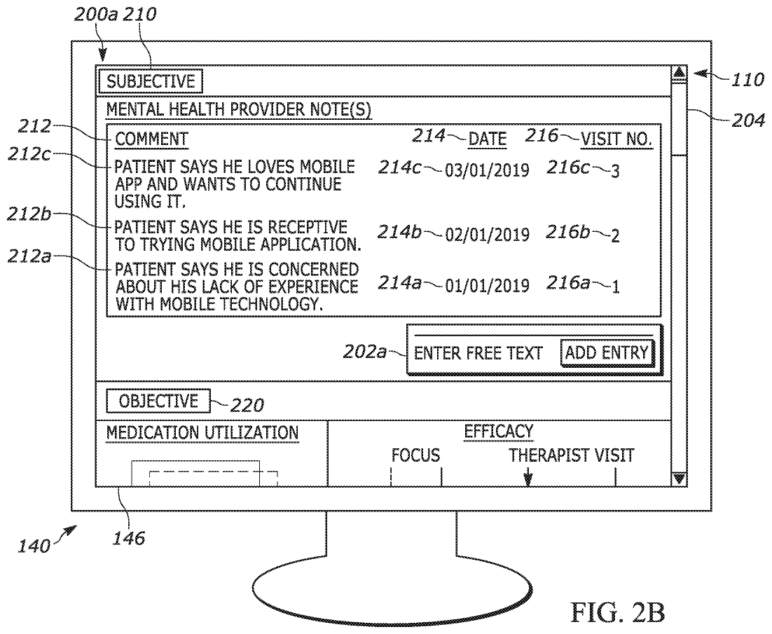

In some implementations, the HCP system 140 (i.e., via the data processing hardware 142) executes a HCP application 110 (or accesses a web-based patient application) for establishing a connection with the therapy service 160 to input and retrieve data therefrom. For instance, the HCP system 140 may be able to access the anonymized patient record 105 securely stored by the therapy service 160 on the storage resources 156 by providing an authentication token 108 validating that the HCP 109 is supervising the patient 101 and authorized to access the corresponding patient record 105. The authentication token 108 may identify the particular patient 101 associated with the patient record 105 that the HCP system 140 is permitted to obtain from the therapy service 160. The patient record 105 may include time-stamped event data 122 indicating the patient's interaction with the prescription digital therapeutic 120 through the patient application 103 executing on the patient device 102. The HCP application 110, when executing on the data processing hardware 142 of the HCP system 140, is configured to display a variety of graphical user interfaces (GUIs) (e.g., an HCP GUI 200 as shown in FIG. 2A) on the display 146 of the HCP system 140 that, among other things, allow the HCP 109 to input event data 122 associated with particular feelings the patient is experiencing, solicit information from the patient 101, and input clinical notes, e.g., SOAP notes, associated with the patient 101.

In some implementations, the HCP application 110 is in communication with a single patient application 103 for a single patient 101 and manages data associated with the single patient application 103. In other implementations, the HCP application 110 is in communication with several patient applications 103 associated with several patients 101, and the HCP application 110 may manage and display the data associated with the several patient applications 103 in any suitable manner, e.g., by toggling between different views and/or displaying certain data simultaneously. In other implementations, the HCP application 110 is in communication with multiple patient applications 103 for the same patient 101 and simultaneously manages data associated with the multiple patient applications 103. In this implementation, the data from multiple patient applications 103 may be displayed simultaneously in any suitable manner or the data from each patient application 103 may be displayed discretely such that the HCP 109 and/or a mental health provider (MHP) 172 is able to toggle between the discretely-displayed data.

The MHP system 170 may include components (e.g., hardware, software, etc.) substantially similar to the HCP system 140, and, as such, the components of the MHP system 170 are not described in detail. The MHP system 170 may be administered by the MHP 172 including, but not limited to, a therapist, a psychiatrist, a psychologist, a counselor, etc. The MHP system 170 may communicate with the HCP system 140 via the network 106 to provide the HCP system 140 with, e.g., mental health data associated with the patient 101.

The cloud computing resources 150 may be a distributed system (e.g., remote environment) having scalable/elastic resources 152. The resources 152 include computing resources 154 (e.g., data processing hardware) and/or the storage resources 156 (e.g., memory hardware). The cloud computing resources 150 execute the therapy service 160 for facilitating communications with the patient device 102 and the HCP system 140 and storing data on the storage resources 156 within the data storage 158. In some examples, the therapy service 160 and the data storage 158 reside on a standalone computing device. The therapy service 160 may provide the patient 101 with the patient application 103 (e.g., a mobile application, a web-site application, or a downloadable program that includes a set of instructions) executable on the data processing hardware 112 and accessible through the network 106 via the patient device 102 when the patient 101 provides a valid access code 104. Similarly, the therapy service 160 may provide the HCP 109 with the HCP application 110 (e.g., a mobile application, a web-site application, or a downloadable program that includes a set of instructions) executable on the data processing hardware 142 and accessible through the network 106 via the HCP system 140.

Referring to FIGS. 2-5, in some implementations, the HCP application 110 may be executed on the HCP system 140 to display the HCP GUI 200 on the display 146. In some implementations, the HCP GUI 200 may include features associated with a SOAP note for a patient 101. For example, the HCP GUI 200 may include a subjective header 210, an objective header 220, an assessment header 230, and a plan header 240. In some implementations, the HCP GUI 200 may include more, less, and/or different headers associated with a SOAP note for a patient 101, and the headers may be arranged and displayed in any suitable order, including, but not limited to, APSO, OSAP, etc. In other implementations, the HCP GUI 200 may include features associated with forms of documentation other than a SOAP note, such as, for example, psychotherapy notes; progress notes; Supplementary data base information, Observations, Activities, Impressions, Goals, and Plans (SOAIGP) notes; Data, Action, and Plan (DAP) notes; Data, Action, Response, and Plan (DARP) notes; claim clarification notes; etc. The HCP GUI 200 shown in the figures is intended to be illustrative only, and it should be understood that modifications may be made to the HCP GUI 200, such as, for example, modifications to the design, layout, style, size, color, etc.

In some implementations, the display 146 of the HCP system 140 includes a touch screen displaying the HCP GUI 200. In other implementations, the HCP system 140 includes peripherals that allow the HCP 109 to interact with the HCP application 110, such peripherals including, but not limited to, the keyboard 148, a mouse, microphones, speakers, a camera, etc. The data processing hardware 142 may execute GUI software adapted to facilitate interaction with the HCP GUI 200. Described in greater detail below, the HCP 109 may provide user-selections indicating selection to interact with the HCP GUI 200. As used herein, a user-selection may be directed to a user interface (UI) control that includes any displayed element or component of the HCP GUI 200 displayed on the display 146. As such, user-selection indicating selection of a UI control may permit the HCP 109 to provide input, view data, and/or otherwise interact with the HCP GUI 200. Example UI controls include buttons, drop down menus, menu items, tap-and-hold functionality, etc.

Referring to FIG. 2A, the subjective header 210 may be selected, e.g., via a user-selection, and displayed on the display 146. A plurality of GUI elements associated with the subjective header 210 may be displayed on the display 146. For example, information associated with “Mental Health Provider Note(s)” may be displayed, including, but not limited to, comments 212, 212a-c, corresponding dates 214, 214a-c, corresponding visit number 216, 216a-c, etc. In some implementations, the comments 212 may be personal thoughts or feelings spoken or indicated by the patient 101 and documented by the HCP 109 on the HCP application 110 from the perspective of the HCP 109. In other implementations, the comments 212 may be direct quotes from the patient 101 documented by the HCP 109. In still other implementations, the comments 212 may pertain to the physical or emotional outward appearance of the patient 101 from the perspective of the HCP 109. For example, the HCP 109 may document that the patient 101 appears nervous, uneasy, confused, happy, sad, angry, etc.

In some implementations an add entry element 202a may be associated with the subjective header 210. The add entry element 202a may be selected, e.g., via a user-selection, to add a comment 212 and associated data (e.g., date 214, visit number 216, etc.) to the HCP application 110 under the subjective header 210. In some implementations, selection of the add entry element 202a may prompt a new window to display in the HCP GUI 200, enabling the HCP 109 to enter data (e.g., free text data, pre-programmed data, etc.) corresponding to, e.g., a comment 212, a date 214, a visit number 216, etc. Additionally or alternatively, the add entry element 202a may enable the HCP 109 to enter notes in any suitable format. The notes may be stored in association with the SOAP note for a given patient 101.

Referring to FIG. 2B, another exemplary embodiment of the HCP GUI 200a may display the data and GUI elements associated with each of the subjective header 210, the objective header 220, the assessment header 230, and the plan header 240 on a single, continuous page. For example, the HCP GUI 200a may include a scroll bar 204 that enables the HCP 109 to scroll through the data associated with each of the headers.

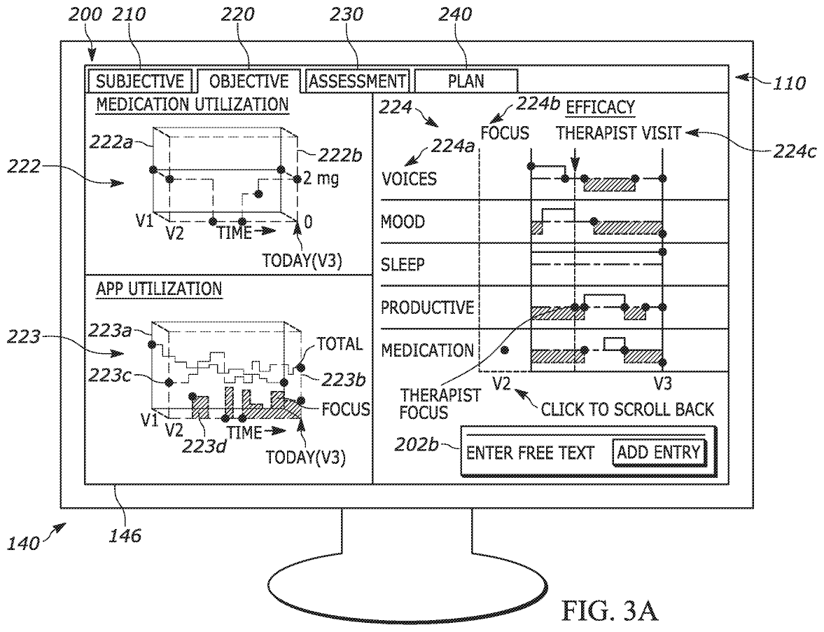

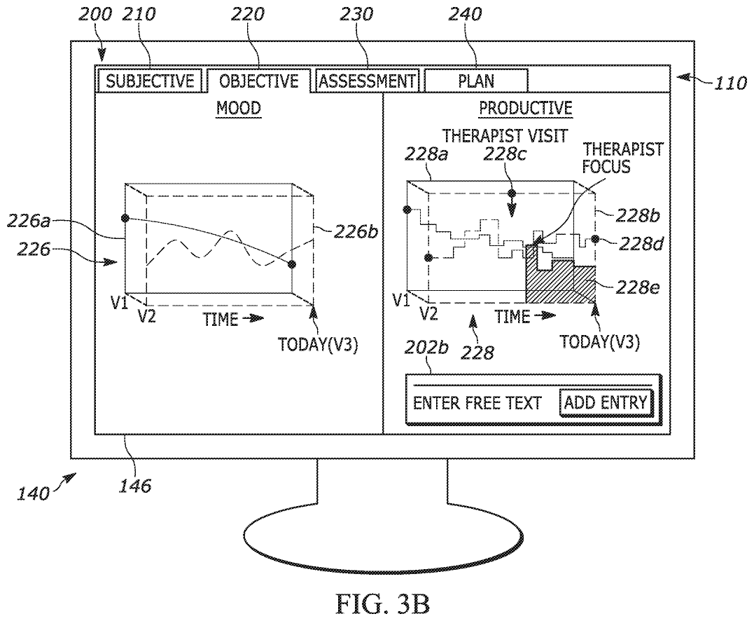

Referring to FIGS. 3A and 3B, the objective header 220 may be selected, e.g., via a user-selection, and displayed on the display 146. A plurality of GUI elements associated with the objective header 220 may be displayed on the display 146. For example, a medication utilization graph 222, a mobile app utilization graph 223, an efficacy graph 224, a mood graph 226, and a productive graph 228 may be displayed. In some implementations, additional, fewer, and/or different GUI elements associated with the objective header 220 may be displayed on the display 146. For example, FIG. 3A may illustrate one example of GUI elements associated with the objective header 220 and FIG. 3B may illustrate another example of GUI elements associated with the objective header 220.

In some implementations an add entry element 202b may be associated with the objective header 220. The add entry element 202b may be selected, e.g., via a user-selection, to add objective data to the HCP application 110 under the objective header 220. In some implementations, selection of the add entry element 202b may prompt a new window to display in the HCP GUI 200, enabling the HCP 109 to enter data (e.g., free text data, pre-programmed data, etc.) corresponding to, e.g., vital signs, physical exam findings, laboratory data, medication adherence, etc. Additionally or alternatively, the add entry element 202b may enable the HCP 109 to enter notes in any suitable format. Again, the notes may be stored in association with the SOAP note for a given patient 101.

In some implementations, the medication utilization graph 222 may include a first panel 222a offset and behind a second panel 222b, such that a portion of the first panel 222a (e.g., the bottom right portion of the first panel 222a) is visually obstructed by the second panel 222b, and a portion of the first panel 222a (e.g., the left and top portions of the first panel 222a) is visually unobstructed by the second panel 222b. The first panel 222a may illustrate the amount of medication the patient 101 utilized between a first visit (“V1”) to the HCP 109 and a second visit (“V2”) to the HCP 109. As can be seen in FIG. 3A, the first panel 222a illustrates that the patient 101 consistently utilized 2 mg of their medication between the first visit and the second visit. The second panel 222b may illustrate the amount of medication the patient 101 utilized between the second visit (“V2”) and a third visit (“V3,” also referred to as “today”). As can be seen in FIG. 3A, the second panel 222b illustrates that, between the second visit and the third visit, the patient 101 initially utilized 2 mg of their medication, then stopped utilizing their medication, then utilized approximately 1 mg of their medication, then utilized 2 mg of their medication.

The period of time between a first visit and a second visit and between the second visit and a third visit may be any suitable duration, e.g., days, weeks, months, years, etc. Additionally, the period of time between the first visit and the second visit may be equal to, greater than, or less than the period of time between the second visit and the third visit. In some implementations, the panels, e.g., the first panel 222a and the second panel 222b, may be organized based on milestones other than visits to the HCP 109. For example, the milestones may include, but are not limited to, days, weeks, months, years, start or end of a medication, start or end of a PDT, start or end of a treatment program, etc.

The orientation of the panels of a graph, i.e., a first panel being offset and behind a second panel, may provide the HCP 109 with an efficient and effective way of tracking the progress of the patient 101 for specific areas of interest, e.g., medication utilization. Additionally, the orientation of the panels may conserve processing resources associated with viewing multiple sets of data in separate windows, screens, files, etc., by enabling the HCP application 110 to instead display multiple sets of data in fewer windows (e.g., one window), in the stacked/offset configuration as described herein. Such advantages are particularly realized when the HCP 109 is managing a large, and potentially overbearing, amount of data, and the HCP application's 110 capabilities of conserving processing resources results in improving the functioning of the computer and improving the efficiency at which the HCP 109 manages the data. Specifically, processing resources may be conserved by reducing at least one of the amount of windows and/or screens displayed by the HCP application 110, the amount of files accessed by the HCP application 110, and/or the amount of clicks required to access and/or manage the data.

In some implementations, there may be more panels, e.g., associated with more visits, and the HCP 109 may be able to scroll through the panels and input user-selections to view specific panels or groupings of panels as desired. In some implementations, the HCP 109 may input user-selections indicating selection of one of the panels, and then manipulate the data displayed by the selected panel. For example, the HCP 109 may add, delete, zoom in or out, rearrange, or otherwise manipulate the data in any suitable manner. In some implementations, the panels may be arranged in any suitable manner to display the progress of the patient 101 between visits. For example, the panels may be arranged in an overlay configuration, a side-by-side configuration, a top-and-bottom configuration, etc. In some implementations, some of the panels may exhibit a suitable level of transparency to aid in viewing the other panels. For example, the first panel may be partially transparent or the second panel may be partially transparent, such that the rear panel (first or second panel) may be partially obstructed by the front panel (first or second panel) and suitable transparency of the front panel allows at least a portion of the rear panel to be viewed through the front panel. In some implementations, the x-axis and the y-axis of the graph may represent any suitable variable and may include any suitable range, e.g., for medication utilization, the x-axis may be time and the y-axis may be milligrams.

In some implementations, the app utilization graph 223 includes a first panel 223a offset and behind a second panel 223b. The first panel 223a may illustrate the amount of time the patient 101 utilized the patient application 103 between a first visit (“V1”) to the HCP 109 and a second visit (“V2”) to the HCP 109. The second panel 222b may illustrate the amount of time the patient 101 utilized the patient application 103 between the second visit (“V2”) and a third visit (“V3,” also referred to as “today”). The second panel 223b may include a total plot 223c and a focus plot 223d. The total plot 223c may illustrate the total amount of time the patient 101 utilized the patient application 103 between the second visit and the third visit, and the focus plot 223d may illustrate the amount of time the patient 101 utilized a specific aspect of the patient application 103 that the HCP 109 and/or the MHP 172 directed the patient 101 to focus on. For example, the patient application 103 may include a variety of modules, including, but not limited to, tasks, assignments, skill areas, etc., and the HCP 109 and/or MHP 172 may direct the patient 101 to focus on at least one of these modules as part of a treatment plan for the patient 101. In some embodiments, the first panel 223a may similarly include the total plot 223c and the focus plot 223d, as applicable.

In some implementations, the efficacy graph 224 includes a module column 224a, a focus column 224b, and a data column 224c. The module column 224a may include a plurality of modules including, but not limited to, “Voices,” “Mood,” “Sleep,” “Productive,” and “Medication,” as shown in FIG. 3A. These modules may be associated with a patient application 103, e.g., for treating schizophrenia. The focus column 224b indicates whether the HCP 109 and/or MHP 172 has directed the patient 101 to focus on that specific module as part of a treatment plan for the patient 101, as set forth above. In one example, the HCP 109 and/or MHP 172 may direct the patient 101 to focus on the “Medication” module, as indicated by the marker in the focus column 224b shown in FIG. 3A. The data column 224c includes data associated with each module between a second visit (“V2”) and a third visit (“V3”). The HCP 109 may interact with the efficacy graph 224, e.g., via user-selections, to display an earlier time period, i.e., between a first visit and the second visit, or to display a later time period as applicable, i.e., between the third visit and a fourth visit. The data column 224c may also include indicators indicating when the patient 101 visited the MHP 172 and/or when and in which module the MHP 172 directed the patient 101 to focus on for the future.

Referring to FIG. 3B, the mood graph 226 may include a first panel 226a offset and behind a second panel 226b. Similar to the aforementioned examples, the panels 226a, 226b of the mood graph 226 may respectively illustrate the quality of mood of the patient 101 over two periods of time, e.g., between a first visit and a second visit and between the second visit and a third visit. The productive graph 228 may include a first panel 228a offset and behind a second panel 228b. In some implementations, the productive graph 228 may include a therapist visit indicator 228c, a total plot 228d, and a therapist focus plot 228e. In some implementations, the therapist visit indicator 228c may be selected, e.g., via a user-selection, to change the display or toggle the display to show the productive graph 228 from the perspective of the MHP 172. For example, the productive graph 228 shown in FIG. 3B may be viewed from the perspective of the HCP 109 and there may be additional, less, and/or different data presented to the HCP 109 compared to the data presented to the MHP 172.

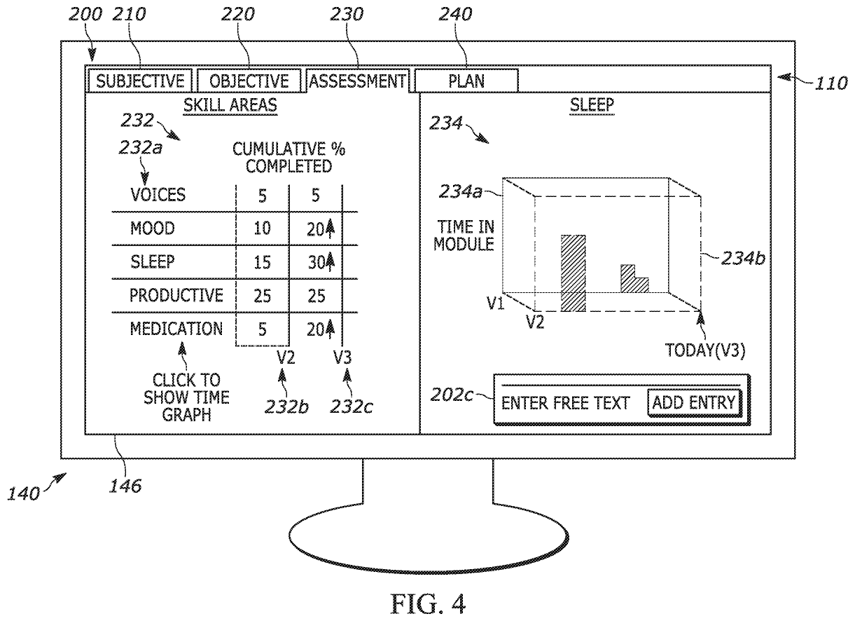

Referring to FIG. 4, the assessment header 230 may be selected, e.g., via a user-selection, and displayed on the display 146. A plurality of GUI elements associated with the assessment header 230 may be displayed on the display 146. For example, a skill area chart 232 and a corresponding sleep graph 234 may be displayed. In some implementations, additional, fewer, and/or different GUI elements associated with the assessment header 230 may be displayed on the display 146.

In some implementations an add entry element 202c may be associated with the assessment header 230. The add entry element 202c may be selected, e.g., via a user-selection, to add assessment data to the HCP application 110 under the assessment header 230. In some implementations, selection of the add entry element 202c may prompt a new window to display in the HCP GUI 200, enabling the HCP 109 to enter data (e.g., free text data, pre-programmed data, etc.) corresponding to, e.g., a diagnosis, an analysis, an assessment of the patient's 101 status, etc. Additionally or alternatively, the add entry element 202c may enable the HCP 109 to enter notes in any suitable format. Yet again, the notes may be stored in association with the SOAP note for a given patient 101.

In some implementations, the skill area chart 232 may include a module column 232a, a first percent completed column 232b, and a second percent completed column 232c. The module column 232a may include a plurality of modules including, but not limited to, “Voices,” “Mood,” “Sleep,” “Productive,” and “Medication,” as shown in FIG. 4. These modules may be associated with a patient application 103, e.g., for treating schizophrenia. The first percent completed column 232b may illustrate the cumulative percent completed for each specific module by the patient 101 between a first visit (“V1”) and a second visit (“V2”). The second percent completed column 232c may illustrate the cumulative percent completed for each specific module by the patient 101 between the second visit (“V2”) and a third visit (“V3”). For example, the “Sleep” module illustrates that the patient completed 15 percent of the module between the first visit and the second visit, and completed another 15 percent of the module between the second visit and the third visit, as indicated by the 30 cumulative percentage in the second percent completed column 232c. In some implementations, the skill area chart 232 may include arrows pointing up or down to indicate that the cumulative percent completed has either increased or decreased, respectively.

Upon the HCP 109 selecting, e.g., via a user-selection, one of the modules, a separate window or screen may open displaying a time graph associated with the selected module. For example, if the HCP 109 selects the “Sleep” module, then the sleep graph 234 may be displayed. The sleep graph 234 may include a first panel 234a offset and behind a second panel 234b. As can be seen by comparing the first panel 234a to the first percent completed column 232b and the second panel 234b to the second percent completed column 232c, the first panel 234a illustrates that the patient completed 15 percent of the module between the first visit and the second visit, and the second panel 234b illustrates that the patient 101 completed another 15 percent of the module between the second visit and the third visit.

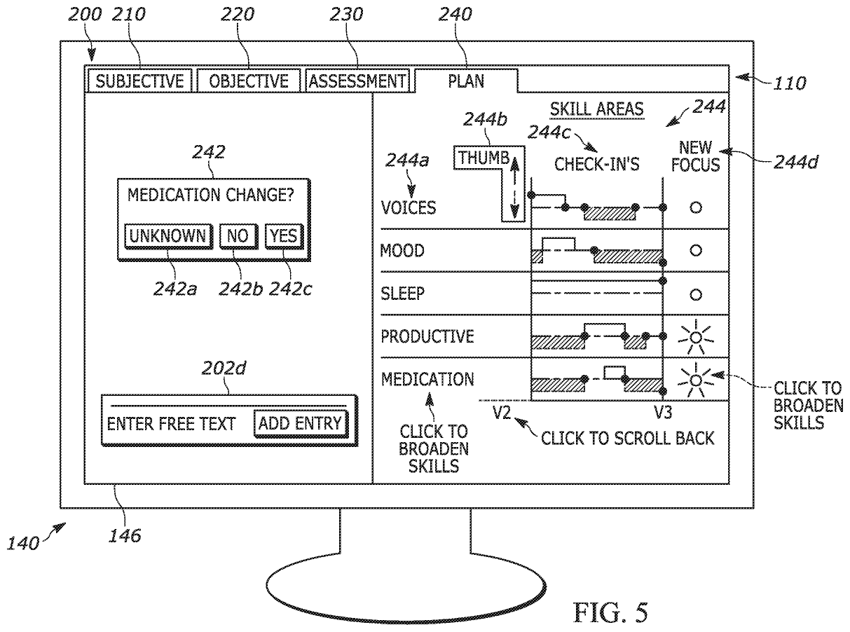

Referring to FIG. 5, the plan header 240 may be selected, e.g., via a user-selection, and displayed on the display 146. A plurality of GUI elements associated with the plan header 240 may be displayed on the display 146. For example, a change medication window 242 and a skill area chart 244 may be displayed. In some implementations, additional, fewer, and/or different GUI elements associated with the plan header 240 may be displayed on the display 146. The elements, functionalities, components, etc., associated with the plan header 240 may be used by the HCP 109 to modify a treatment of the patient 101.

In some implementations an add entry element 202d may be associated with the plan header 240. The add entry element 202d may be selected, e.g., via a user-selection, to add plan data to the HCP application 110 under the plan header 240. In some implementations, selection of the add entry element 202d may prompt a new window to display in the HCP GUI 200, enabling the HCP 109 to enter data (e.g., free text data, pre-programmed data, etc.) corresponding to, e.g., concerns, considerations, possible medication changes, etc. Additionally or alternatively, the add entry element 202d may enable the HCP 109 to enter notes in any suitable format. The notes may be stored in association with the SOAP note for a given patient 101.

The change medication window 242 may include an unknown button 242a, a no button 242b, and a yes button 242c. The HCP 109 may select, e.g., via a user-selection, one of these buttons to indicate whether the medication for the patient 101 should be changed. Such a change may include the type of medication, the dosage of medication, the means for administering the medication, etc. The determination of whether to change the patient's medication may be completed by the HCP 109 or artificial intelligence and/or machine learning. Such a determination may be based on at least the event data 122 and/or any content in the SOAP note(s).

The skill area chart 244 may include a module column 244a, an input button 244b, a check-in column 244c, and a new focus column 244d. The module column 244a may include a plurality of modules including, but not limited to, “Voices,” “Mood,” “Sleep,” “Productive,” and “Medication,” as shown in FIG. 5. These modules may be associated with a patient application 103, e.g., for treating schizophrenia. The HCP 109 may interact with the input button 244b, e.g., via user-selections, to increase or decrease the emphasis placed on each module for the treatment plan for the patient 101. The check-in column 244c may illustrate the amount of times the patient 101 checked in to each specific module in the patient application 103. This data may provide the HCP 109 and/or the MHP 172 with a basis for determining which modules to direct the patient 101 to focus on for the future.

The new focus column 244d illustrates modules that the HCP 109 and/or the MHP 172 has directed the patient 101 to focus on. In some implementations, the new focus column 244d may include indications of recommended modules to focus on as recommended by the HCP application 110. For example, the HCP application 110 may include artificial intelligence and/or machine learning (supervised or unsupervised) to display recommendations on the display 146 of which modules to direct the patient 101 to focus on for the future. Such recommendations may be based on, for example, weighted or unweighted analysis of historical data concerning, inter alia, information about a particular patient's interaction with the patient application 103 (i.e., patient-specific data), information about a plurality of different patients' interactions with respective patient applications 103 (i.e., multi-patient data), and/or some combination of patient-specific data and multi-patient data.

Referring to Table 1 below, the sources of the previously described data is illustrated.

TABLE 1
Sources of Data Managed by HCP Application.

| Treatment | Adherence | Efficacy | Customization |
|---|---|---|---|
| Medication | Patient | Patient, HCP | HCP |
| PDT | App | App, Patient, | HCP, MHP |
| | | HCP, MHP | |
| Synergy | HCP | HCP | HCP |
| SOAP | Prescription | HCP | Mental Health |
| | Digital | Application | Provider (MHP) |
| | Therapeutic (PDT) | | |
| Subjective (S) | Possible | Possible | Patient-reported |
| | | | medication |
| | | | adherence and |
| | | | efficacy |
| Objective (O) | App (adherence, | MHP (adherence, | Yes (mental status |
| | check-in, sensor | focus, sensor | exam, lab data) |
| | data, lab data) | data, lab data) | |
| Assessment (A) | No | No | Summary efficacy; |
| | | | synergy |
| Plan (P) | No | Yes (recommend | Start/stop PDT, |
| | | focus) | change medication, |
| | | | change PDT |

As can be seen above, data sourced from the patient 101 may be derived from the patient interacting with the patient application 103, which may be referred to as active data. Additionally, the patient application 103 may collect passive data about the patient 101. For example, such passive data may include the amount of times the patient 101 has checked in to the patient application 103 and/or a specific module on the patient application 103, the amount of time the patient 101 has spent on the patient application 103 and/or a specific module on the patient application 103, etc. Data sourced from the HCP 109 may be derived from the HCP 109 interacting with the HCP application 110. Data sourced from the MHP 172 may be derived from the MHP 172 interacting with the MHP application 170. Data sourced from the patient application 103 and HCP application 110 may be derived from sensor data (e.g., generated by the one or more sensors 180) or lab data (e.g., blood sample, urine sample, etc.). Data sourced from the MHP application 170 may likewise be derived from lab data.

In some implementations, as set forth above, artificial intelligence and/or machine learning may be utilized for various features, functions, components, processes, modules, etc., of the HCP application 110 (e.g., free text entry, change medication, etc.). For example, free text data entered via the add entry element 202 may be leveraged by implementing fuzzy matching to compare the free text data to entries in an externally available database and/or a local database. This analysis of the particular free text data may be reviewed by humans (e.g., clinicians, healthcare providers, third-party services, etc.), artificial intelligence, machine learning, etc., to verify a match between the free text data and an entry in a database. As another example, medication changes (e.g., via the change medication window 242) may implement artificial intelligence to determine whether to change the patient's medication. This medication change may similarly be supervised or unsupervised.

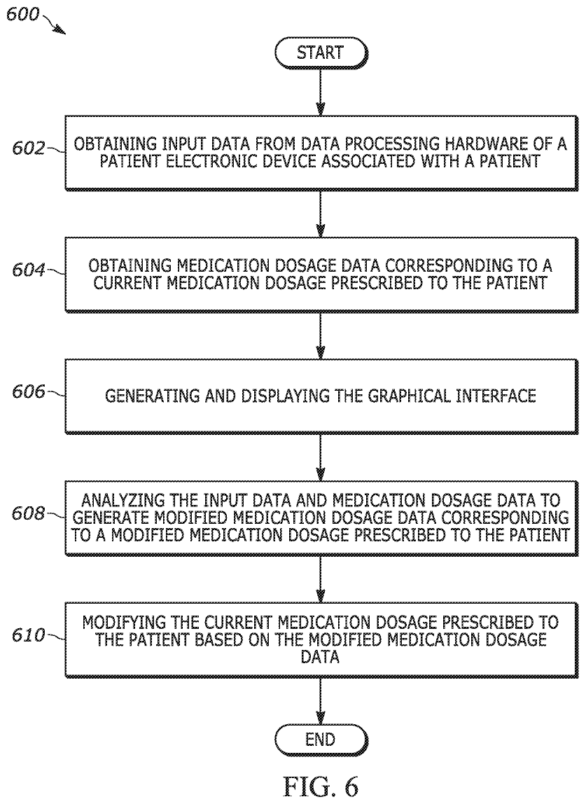

Referring to FIG. 6, a flowchart illustrating a method 600 implementing the systems and processes described herein is generally shown. At 602, input data (e.g., the event data 122) is obtained, via one or more processors (e.g., the data processing hardware 142), from data processing hardware (e.g., the data processing hardware 112) of a patient electronic device (e.g., the patient device 102) associated with a patient (e.g., the patient 101). At 604, medication dosage data corresponding to a current medication dosage prescribed to the patient is obtained via the one or more processors. At 606, a graphical interface (e.g., the HCP GUI 200) is generated and displayed on a display (e.g., the display 146) via the one or more processors. At 608, the input data and the medication dosage data are analyzed, via the one or more processors, to generate modified medication dosage data corresponding to a modified medication dosage prescribed to the patient. At 610, the current medication dosage prescribed to the patient is modified, via the one or more processors, based on the modified medication dosage data. It should be understood that the foregoing steps of the method 600 may include additional or fewer steps, and the steps may be performed in any suitable order.

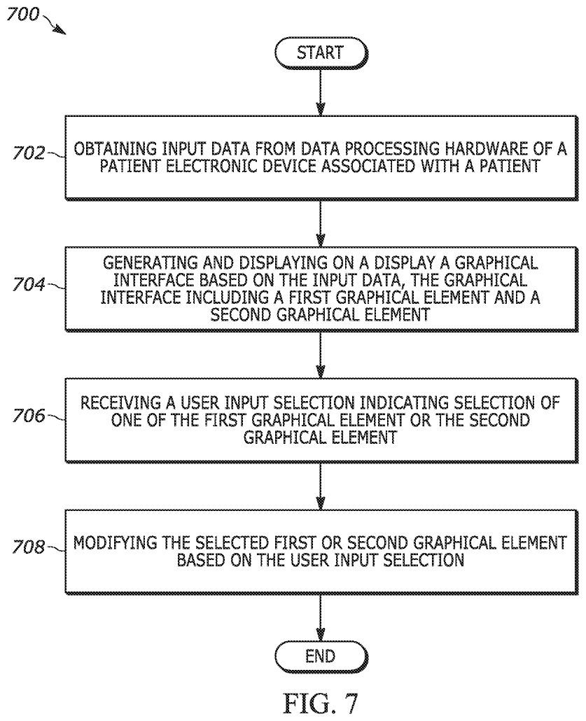

Referring to FIG. 7, a flowchart illustrating a method 700 implementing the systems and processes described herein is generally shown. At 702, input data (e.g., the event data 122) is obtained, via one or more processors (e.g., the data processing hardware 142), from data processing hardware (e.g., the data processing hardware 112) of a patient electronic device (e.g., the patient device 102) associated with a patient (e.g., the patient 101). At 704, a graphical interface (e.g., the HCP GUI 200) is generated and displayed on a display (e.g., the display 146) via the one or more processors, the graphical interface including a first graphical element (e.g., the panel 222b) and a second graphical element (e.g., the panel 222a). At 706, a user input selection indicating selection of one of the first graphical element or the second element is received, via the one or more processors. At 708, the selected first or second graphical element is modified, via the one or more processors, based on the user input selection. It should be understood that the foregoing steps of the method 700 may include additional or fewer steps, and the steps may be performed in any suitable order.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

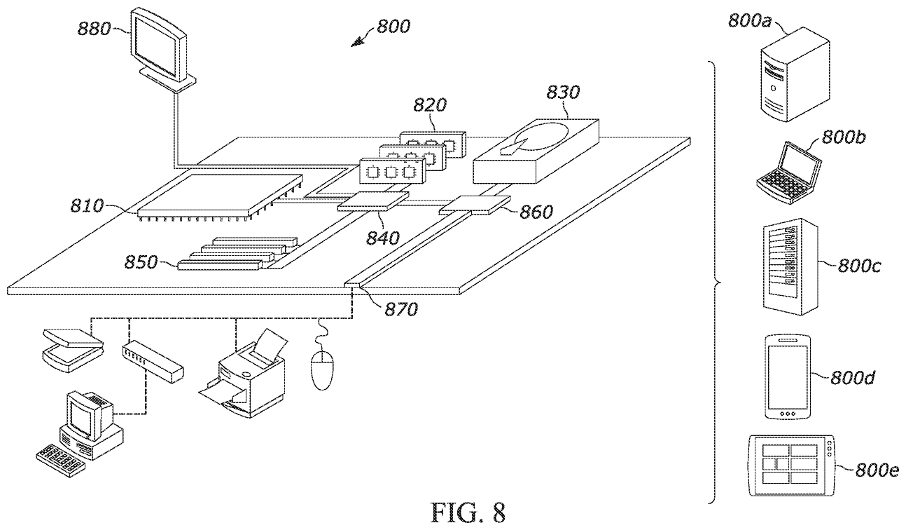

FIG. 8 is schematic view of an example computing device 800 that may be used to implement the systems and methods described in this document. The computing device 800 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 800 includes a processor 810, memory 820, a storage device 830, a high-speed interface/controller 840 connecting to the memory 820 and high-speed expansion ports 850, and a low speed interface/controller 860 connecting to a low speed bus 870 and a storage device 830. Each of the components 810, 820, 830, 840, 850, and 860, are interconnected using various busses, and may be mounted on a common motherboard or in other manners as appropriate. The processor 810 can process instructions for execution within the computing device 800, including instructions stored in the memory 820 or on the storage device 830 to display graphical information for a graphical user interface (GUI) on an external input/output device, such as display 880 coupled to high speed interface 840. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 820 stores information non-transitorily within the computing device 800. The memory 820 may be a computer-readable medium, a volatile memory unit(s), or non-volatile memory unit(s). The non-transitory memory 820 may be physical devices used to store programs (e.g., sequences of instructions) or data (e.g., program state information) on a temporary or permanent basis for use by the computing device 800. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 830 is capable of providing mass storage for the computing device 800. In some implementations, the storage device 830 is a computer-readable medium. In various different implementations, the storage device 830 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 820, the storage device 830, or memory on processor 810.

The high speed controller 840 manages bandwidth-intensive operations for the computing device 800, while the low speed controller 860 manages lower bandwidth-intensive operations. Such allocation of duties is exemplary only. In some implementations, the high-speed controller 840 is coupled to the memory 820, the display 880 (e.g., through a graphics processor or accelerator), and to the high-speed expansion ports 850, which may accept various expansion cards (not shown). In some implementations, the low-speed controller 860 is coupled to the storage device 830 and a low-speed expansion port 890. The low-speed expansion port 890, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 800 may be implemented in a number of different forms, as shown in FIG. 8. For example, it may be implemented as a standard server 800a or multiple times in a group of such servers 800a, as a laptop computer 800b, as part of a rack server system 800c, as a smartphone 800d, as a tablet device 800e, etc.

Other benefits to the HCP application 110 include, but are not limited to: interfacing with PDTs; supporting the HCP 109 and/or the MHP 172 who are about to or have prescribed a PDT; assisting the HCP 109 and/or the MHP 172 in reviewing reported symptoms of the patient 101; assisting the HCP 109 and/or the MHP 172 in reviewing medication adherence information; delivering patient 101 and/or MHP 172 PDT utilization information to the HCP 109; delivering MHP 172 assessment of efficacy to the HCP 109; allowing the HCP 109 to record assessment of medication, PDT, MHP 172 efficacy, individually and in synergistic combination; allowing the HCP 109 and/or the MHP 172 to customize PDT for each patient 101 according to the clinical judgment of the HCP 109 and/or the MHP 172; delivering therapeutic content to the HCP 109 and/or the MHP 172 to support use of the PDT; displaying patient-related information, such as medication and PDT utilization; supporting the HCP 109 and others who are involved in the provision of the care of patients 101; organizing information relative to each encounter (e.g., a clinic visit) of a specific HCP 109 and/or MHP 172 with the specific patient 101, thus, enabling the HCP 109 and/or the MHP 172 to view and comprehend events in relation to his/her own frame of reference; and allowing the HCP 109 and/or the MHP 172 to change the time frame of the display interactively, e.g., to view data along a timeline.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Among other advantages, the systems and methods of the present disclosure facilitate a conservation of processing resources by compressing visual data in a manner so as to relay the same information using fewer bits. Other advantages include improving the functioning of the computer, making the computer operate more efficiently, and solving the technical problem of managing large amounts of data.

A number of implementations have been described. Nevertheless, it will be understood that various modifications may be made without departing from the spirit and scope of the disclosure. Accordingly, other implementations are within the scope of the following claims.

What is claimed is:

1. A system comprising:
data processing hardware; and
memory hardware in communication with the data processing hardware, the memory hardware storing instructions that when executed on the data processing hardware cause the data processing hardware to perform operations comprising:
obtaining input data from data processing hardware of a patient electronic device associated with a patient, the input data corresponding to a subjective, objective, assessment, and plan (SOAP) note;
obtaining medication dosage data corresponding to a current medication dosage prescribed to the patient;
generating and displaying on a display a graphical interface based on the input data and the medication dosage data, the graphical interface comprising:
a first graphical element displaying a first subset of the input data, the first subset of the input data corresponding to a first duration of time; and
a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed;
analyzing the input data and the medication dosage data to generate modified medication dosage data corresponding to a modified medication dosage prescribed to the patient; and
modifying the current medication dosage prescribed to the patient based on the modified medication dosage data.

2. The system of claim 1, wherein the input data includes active data input to the patient electronic device by the patient and passive data collected by the patient electronic device.

3. The system of claim 1, wherein the input data includes at least one of medication utilization data, application utilization data, efficacy data, mood data, productivity data, skill areas data, or sleep data.

4. The system of claim 1, wherein the operations further comprise:
analyzing the input data and the medication dosage data to generate change medication data corresponding to a change of the medication prescribed to the patient; and
modifying the current medication dosage prescribed to the patient based on the change medication data.

5. The system of claim 1, wherein at least a portion of the input data is generated by a healthcare provider associated with the patient.

6. The system of claim 1, wherein the modified medication dosage data is generated by implementing artificial intelligence.

7. The system of claim 1, wherein the graphical interface further comprises an add entry element configured to facilitate entry of free text data by a healthcare provider associated with the patient.

8. The system of claim 1, wherein the patient electronic device is one of a desktop computer, a laptop computer, a tablet computer, a smartphone, or a wearable device.

9. A system comprising:
data processing hardware; and
memory hardware in communication with the data processing hardware, the memory hardware storing instructions that when executed on the data processing hardware cause the data processing hardware to perform operations comprising:
obtaining input data from data processing hardware of a patient electronic device associated with a patient;
generating and displaying on a display a graphical interface based on the input data, the graphical interface comprising:
a first graphical element displaying a first subset of the input data, the first subset of the input data corresponding to a first duration of time; and
a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed;
receiving a user input selection indicating selection of one of the first graphical element or the second graphical element; and
modifying the selected first or second graphical element based on the user input selection.

10. The system of claim 9, wherein the input data includes active data input to the patient electronic device by the patient and passive data collected by the patient electronic device.

11. The system of claim 9, wherein the input data includes at least one of medication utilization data, application utilization data, efficacy data, mood data, productivity data, skill areas data, or sleep data.

12. The system of claim 9, wherein the input data corresponds to a subjective, objective, assessment, and plan (SOAP) note.

13. The system of claim 12, wherein at least a portion of the input data is generated by a healthcare provider associated with the patient.

14. The system of claim 13, wherein at least a portion of the input data is generated by a mental health provider associated with the patient.

15. The system of claim 9, wherein the graphical interface further comprises an add entry element configured to facilitate entry of free text data by a healthcare provider associated with the patient.

16. A method comprising:
obtaining, via one or more processors, input data from data processing hardware of a patient electronic device associated with a patient, the input data corresponding to a subjective, objective, assessment, and plan (SOAP) note;
obtaining, via the one or more processors, medication dosage data corresponding to a current medication dosage prescribed to the patient;
generating and displaying, via the one or more processors, on a display a graphical interface based on the input data and the medication dosage data, the graphical interface comprising:
a first graphical element displaying a first subset of the input data, the first subset of the input data corresponding to a first duration of time; and
a second graphical element displaying a second subset of the input data, the second subset of the input data corresponding to a second duration of time different than the first duration of time, the second graphical element being offset and behind the first graphical element such that (i) a portion of the second graphical element is visible through the first graphical element, (ii) a portion of the second graphical element is visually unobstructed by the first graphical element, and (iii) the entire first graphical element is visually unobstructed;
analyzing, via the one or more processors, the input data and the medication dosage data to generate modified medication dosage data corresponding to a modified medication dosage prescribed to the patient; and
modifying, via the one or more processors, the current medication dosage prescribed to the patient based on the modified medication dosage data.

17. The method of claim 16, wherein the input data includes active data input to the patient electronic device by the patient and passive data collected by the patient electronic device.

18. The method of claim 16, wherein the input data includes at least one of medication utilization data, application utilization data, efficacy data, mood data, productivity data, skill areas data, or sleep data.

19. The method of claim 16, wherein at least a portion of the input data is generated by a healthcare provider associated with the patient.

20. The method of claim 16, wherein the modified medication dosage data is generated by implementing artificial intelligence.
